(19) United States
(12) Patent Application Publication
(10) Pub. No.: US 2025/0250586 A1
(43) Pub. Date: Aug. 7, 2025

(54) Base Editing System for Achieving A-To-C and/or A-To-T Base Mutations and Use Thereof

(71) Applicant: EAST CHINA NORMAL UNIVERSITY, Shanghai (CN)

(72) Inventors: Liang CHEN, Shanghai (CN); Dali LI, Shanghai (CN); Mengjia HONG, Shanghai (CN); Changming LUAN, Shanghai (CN)

(73) Assignee: EAST CHINA NORMAL UNIVERSITY, Shanghai (CN)

(21) Appl. No.: 18/686,437

(22) PCT Filed: Aug. 27, 2021
(86) PCT No.: PCT/CN2021/115084

§ 371 Date: Feb. 26, 2024

(30) Foreign Application Priority Data
Aug. 26, 2021 (CN) 202110988933.6

(51) Int. Cl. C12N 15/90; C12N 9/22; C12N 9/24; C12N 9/78; C12N 15/11

(52) U.S. Cl. CPC C12N 15/90; C12N 9/224; C12N 9/2497; C12N 9/78; C12N 15/11; C12Y 302/02021; C12Y 305/04004; C12N 2310/20

(57) ABSTRACT

A base editing system for achieving A-to-C and/or A-to-T base mutations and a use thereof are provided. A base editor is constructed by means of fusing 3-methyladenine DNA glycosylase with adenosine deaminase and Cas9 nuclease with impaired catalytic activity, which achieves adenine-based transversion for the first time. It is found through experimental comparison that AXBE, which is constructed by means of fusing mouse-derived 3-methyladenine DNA glycosylase with adenosine deaminase TadA-8e derived from E. coli and Cas9 nickase with impaired catalytic activity derived from Streptococcus pyogenes, has the best effect of catalyzing the transversion of adenine. The use of the base editing system in the gene therapy, cell therapy, human disease model production, and crop genetic breeding, etc. is promoted.

### CROSS REFERENCE TO THE RELATED APPLICATIONS

This application is the national phase entry of International Application No. PCT/CN2021/115084, filed on Aug. 27, 2021, which is based upon and claims priority to Chinese Patent Application No. 202110988933.6, filed on Aug. 26, 2021, the entire contents of which are incorporated herein by reference.

### SEQUENCE LISTING

The instant application contains a Sequence Listing which has been submitted in ASCII format via EFS-Web and is hereby incorporated by reference in its entirety. Said ASCII copy is named GBBJQH020-20240304_ST25.txt, created on Mar. 4, 2024, and is 39,281 bytes in size.

### TECHNICAL FIELD

The present disclosure belongs to the field of biotechnologies, and particularly relates to a base editing system for achieving A-to-C and/or A-to-T base mutations and a use thereof.

### BACKGROUND

The essence of human genetic diseases is due to genetic mutations, and about 60% of genetic diseases are caused by the mutation of a single base. Traditional use of genome editing technology mediated homologous recombination to correct such genetic diseases is very inefficient (0.1% to 5%). A single base editor derived based on a CRISPR system is an emerging efficient base editing technology in recent years, and due to the advantages of not producing DNA double strand break, no need for recombination of templates, and efficient editing, it has shown great application prospects in basic research and clinical disease treatment.

Classic base editors are mainly divided into cytosine base editors (CBEs) and adenine base editors (ABEs). The former is composed of spCas9 nickase derived from Streptococcus pyogenes with impaired activity, cytosine deaminase rAPOBEC1 derived from rats, and uracil glycosidase inhibitors, where a Cas9 protein recognizes and specifically binds to DNA using NGG as PAM, and then, under the action of deaminase and DNA repair, finally, the substitution of C⋅G-T⋅A is achieved within a range of 20 bp of an upstream targeting sequence of NGG (positions 21-23), with an editing window mainly located at positions 4-8, which is expected to correct 14% of human pathogenic point mutations. The latter fuses TadA derived from bacteria with spCas9, with the assistance of directed evolution and protein engineering modification technologies, an adenine base editor ABE7.10 that can act on single stranded DNA is finally obtained after 7 rounds of evolution, and an active editing region is mainly located at positions 4-7. The average editing efficiency of A⋅T-G⋅C caused by this system in human cells is about 53%, which is far higher than the efficiency of using homologous recombination to mediate base mutations, the purity of its product is as high as 99.9%, and the occurrence of insertions and deletions (indels) is extremely low. More importantly, about 47% of human pathogenic point mutations is formed by a mutation from C⋅G to T⋅A, while the adenine base editor is expected to correct nearly half of the pathogenic point mutations, demonstrating its enormous potential in mutation base modification and genetic disease treatment. Currently, the ABE has been widely applied to animal model preparation and gene therapy.

Both the CBE and the ABE can only achieve base conversion. In the early stages of developing the CBE, scientists found that knocking out intracellular uracil glycosidase (UNG) or removing uracil glycosidase inhibitors (UGI) would produce C⋅G-to-G⋅C and C⋅G-to-A⋅T editing byproducts, i.e., cytosine-based transversion occurred. Recently, according to the editing byproduct phenomenon generated by the previous CBE, scientists have developed a CGBE series by fusing the CBE with the UGI removed with different types of UNG, DNA damage repair proteins, or translesion polymerase, which is expected to correct 11% of G⋅C to C⋅G pathogenic point mutations.

However, there is no reported enzyme that can directly catalyze adenine (A) in genomic DNA to cytosine (C) or thymine (T), and human pathogenic point mutations that require A-to-C and A-to-T to reverse account for nearly a quarter of human disease-associated point mutations, and especially, transversion of A⋅T to C⋅G with a ratio of 16% can correct the second most common pathogenic SNV, which is beyond the scope of diseases that may be covered by the classic CBE.

### SUMMARY

The present disclosure aims to provide a base editing system for achieving A-to-C and/or A-to-T base mutations and a use thereof. A base editor is constructed by means of fusing 3-methyladenine DNA glycosylase with adenosine deaminase and Cas9 nuclease with impaired catalytic activity, which achieves adenine-based transversion for the first time, including a mutation from A to C and a mutation from A to T.

In order to implement the above objective, a technical solution of the present disclosure is summarized as follows.

A gene editing system for achieving A-to-C and/or A-to-T base mutations includes adenosine deaminase TadA, Cas9 nuclease and 3-methyladenine DNA glycosylase.

Preferably, a gene sequence of the 3-methyladenine DNA glycosylase is as shown in any one of SEQ ID NOS: 1-4, an amino acid sequence of the 3-methyladenine DNA glycosylase is as shown in any one of SEQ ID NOS: 5-8, and more preferably, the 3-methyladenine DNA glycosylase is derived from rats, mice or Bacillus subtilis.

The amino acid sequences or nucleotide sequences involved above, sequences having a homology of above 80%, above 85%, above 90%, above 95%, above 96%, above 97%, above 98%, or above 99% with the sequences involved in this application, and/or sequences obtained after substitution, deletion or insertion of amino acid residues or nucleotides based on the sequences involved in this application, and sequences having the same or similar functions as the sequences involved in this application are all within the scope of protection of this application.

Sources of the adenosine deaminase TadA include E. coli, Staphylococcus aureus, Oceanobacillus sojae and Acinetobacter, and preferably, the adenosine deaminase TadA is derived from the E. coli; and more preferably, the TadA derived from the E. coli is TadA-8e.

The Cas9 nuclease includes spCas9 derived from Saccharomyces cerevisiae, Cas9 nickase, and variants VQR-spCas9, VRER-spCas9, spRY and spNG thereof, and SaCas9 derived from Staphylococcus aureus and variants SaCas9-KKH and SaCas9-NG, and also includes LbCas12a derived from Lachnospiraceae and enAsCas12a derived from Acidaminococcus, the Cas9 nuclease may further be replaced by other nucleases which can specifically recognize DNA and have a cutting function, preferably, the Cas9 nuclease is Cas9 nickase, and preferably, the Cas9 nickase is derived from Streptococcus pyogenes.

The present disclosure further discloses a gene editing method for achieving A-to-C and/or A-to-T base transversion, including the following step:
expressing the adenosine deaminase, the Cas9 nuclease and the 3-methyladenine DNA glycosylase described above in host, so as to perform base editing on a target gene in genome of the host, wherein preferably, the host is eukaryote cells, more preferably, the host is mammalian cells, and more preferably, the host is cell derived from rats, mice or Bacillus subtilis.

The “expressing the adenosine deaminase, the Cas9 nuclease and the 3-methyladenine DNA glycosylase in a host” is expressing a coding gene of the adenosine deaminase, the Cas9 nuclease and the 3-methyladenine DNA glycosylase by introducing the coding gene of the adenosine deaminase, the Cas9 nuclease and the 3-methyladenine DNA glycosylase into eukaryotic cells, so as to achieve A-to-C and/or A-to-T mutations.

More specifically, a specific achieving process of the A-to-C and/or A-to-T base mutations is: under a combined action of the Cas9 nuclease and the adenosine deaminase, adenine of a target sequence in the genome is deaminated into hypoxanthine, the hypoxanthine is recognized and excised through the 3-methyladenine DNA glycosylase, finally, an apurinic/apyrimidinic site is formed at this site, and in the end, transversion of A-to-C and/or A-to-T occurs under the mediation of endogenous DNA damage repair.

In addition, the selection of targets is not limited by targets listed in specific embodiments of the present disclosure, any target that can verify functions of the gene editing system of the present disclosure can be selected, preferably, an editing window range achieving A-to-C and/or A-to-T is mainly located at the positions 2-10 at the 5′ terminus of the target gene (20 base sequences), represented as A2-A10, that is, A located at the base positions 2-10 at the 5′ terminus can achieve transversion of A-to-C and/or A-to-T.

In addition, any product including the above gene editing system also falls within the scope of protection of the present disclosure. The product includes a kit and a pharmaceutical composition, but is not limited to this. Any product applied to the gene editing system of the present disclosure falls within the scope of protection of the present disclosure.

In addition, the cells used in the present disclosure are commonly used HEK293T cells, also including cells derived from human and other mammals, such as Hela, U2OS, NIH3T3, and N2A. The cells also include gametes and fertilized eggs derived from human and other mammals.

The cells used in the present disclosure are eukaryocyte, including non-eukaryocyte such as prokaryotes and palaeobios. The cells also include cells in animal bodies that can achieve editing, treatment, and gene expression regulation.

An AXBE used in the present disclosure is composed of CMV-TadA8e-Cas9 nickase-HDG4-BGH polyA, and also includes arrangements and combinations which can perform more efficient or precise A-to-C and/or A-to-T compared to the AXBE, as well as other positional orientations such as embedding TadA protein in the middle of Cas9.

A promoter element used is CMV, which also includes other types of spectral promoters and tissue-specific promoters, such as CAG, PGK, EF1α, a muscle-specific promoter MHCK7 and a liver-specific promoter Lp1; and polyA used is a bovine growth hormone polyadenylation signal BGH polyA, which also includes other species, including eukaryotic and prokaryotic polyadenylation signals.

The TadA used in the embodiments of the present disclosure is tad derived from E. coli, but is not limited to this, and also includes TadA derived from other species and other prokaryotic organisms.

The advantages of the present disclosure:
the present disclosure discloses the base editing system for achieving the A-to-C and/or A-to-T base mutations for the first time. The base editor is constructed by means of fusing the 3-methyladenine DNA glycosylase with the adenosine deaminase and the Cas9 nuclease with impaired catalytic activity, which achieves adenine-based transversion for the first time. The 3-methyladenine DNA glycosylase with the ability of recognizing and excising the hypoxanthine forms the gene editing system with the adenosine deaminase TadA-8e and the Cas9 nuclease. Under a combined action of the Cas9 nuclease and the adenosine deaminase TadA-8e, adenine of a target sequence in the genome is deaminated into hypoxanthine, the hypoxanthine is excised through the 3-methyladenine DNA glycosylase, finally, an apurinic/apyrimidinic site is formed at this site, and in the end, transversion of A-to-C and A-to-T occurs under the mediation of endogenous DNA damage repair.

In the present disclosure, by comparing DNA glycosylases (HDGs) from different sources, it is found that the AXBE, which is constructed by means of fusing the mouse-derived 3-methyladenine DNA glycosylase with the monomer adenosine deaminase TadA-8e derived from E. coli and the Cas9 nickase nickase with single-chain cutting activity derived from Streptococcus pyogenes, has the best effect of catalyzing the transversion of adenine. The adenine transversion is achieved in mammal cells for the first time, i.e., the mutation from A to C and the mutation from A to T, experiment results show that the highest editing efficiency of A⋅T to C⋅G is 23.4%, the highest editing efficiency of A⋅T to T⋅A is 12%, the AXBE is expected to correct SNP related to 16% of C⋅G to A⋅T disease mutation points or SNP related to 7% of T⋅A to A⋅T disease mutation points, which is a significant technological innovation in the technical field of single base gene editing, and the use of the base editing system in the gene therapy, cell therapy, human disease model production, and crop genetic breeding will also be greatly promoted.

### BRIEF DESCRIPTION OF THE DRAWINGS

FIGS. 1A-1B are a principle of adenine-based transversion, namely a mutation from A-to-C and a mutation from A-to-T.

FIG. 2 is the design of the fusion of nine different HDGs with TadA-8e and Cas9 nickase as well as the design of different position fusion of HDG4.

FIGS. 3A-3B are comparison of achieving adenine editing at target sites PD-1-sg4 and PD-1-sg3 in HEK293T by nine HDG constructions and control ABE8e.

FIGS. 4A-4E are comparison of achieving adenine editing at 5 target sites in HEK293T induced by ABE8e, AH4, AH4-M and AH4-N.

FIG. 5 is a plasmid profile of AXBE.

FIGS. 6A-6F are comparison of achieving adenine editing at 5 target sites in HEK293T induced by ABE8e and AXBE.

### DETAILED DESCRIPTION OF THE EMBODIMENTS

The present disclosure is further described below in conjunction with specific embodiments, and the advantages and characteristics of the present disclosure will be clearer with the description. But specific experimental methods involved in the following embodiments, unless otherwise specified, are all conventional methods or implemented according to the conditions recommended in the manufacturer's instructions.

If not specially specified, the technical means used in the embodiments are conventional means well-known to those skilled in the art. The experimental methods in the following embodiments, unless otherwise specified, are all conventional methods. Unless otherwise specified, reagents and materials adopted can all be purchased from the market.

Unless otherwise defined, all professional and scientific terms used in the text have the same meanings as those familiar to skilled professionals in the art. In addition, any methods and materials similar or equal to the recorded content can be applied to the present disclosure. Preferred implementation methods and materials described in the text are only for demonstration purposes.

Unless otherwise specified, the implementation of the present disclosure will use conventional botanical techniques, microbiology, tissue culture, molecular biology, chemistry, biochemistry, DNA recombination, and bioinformatics techniques that are readily apparent to those skilled in the art. These techniques have been fully explained in publicly available literatures. In addition, methods used in the present disclosure, such as DNA extraction, construction of phylogenetic trees, gene editing methods, construction of gene editing vectors, and acquisition of gene edited animals, can be achieved using methods already disclosed in existing literatures, except for the methods used in the following embodiments.

The terms “nucleic acid”, “nucleic acid sequence”, “nucleotide”, “nucleic acid molecule” or “polynucleotide” used here refer to DNA or RNA molecules composed of isolated DNA molecules (such as cDNA or genomic DNA), RNA molecules (such as messenger RNA), natural types of, mutation types of and synthesized DNA or RNA molecules, and nucleotide analogues, and the DNA or RNA molecules are of single-stranded or double-stranded structures. These nucleic acids or polynucleotides include gene coding sequences, antisense sequences, and regulatory sequences of non-coding regions, but are not limited to these. These terms include one gene. A “gene” or “gene sequence” is widely used to refer to a functional DNA nucleic acid sequence. Therefore, a gene may include an intron and an exon in a genomic sequence, and/or a coding sequence in cDNA, and/or cDNA and its regulatory sequence. In special implementation schemes, such as for an isolated nucleic acid sequence, it is prioritized and assumed to be cDNA by default.

Gene editing is an emerging gene functional technique that precisely modifies specific target sequences of an organism's genome.

“Cell transfection” refers to a technique of introducing exogenous molecules such as DNA and RNA into eukaryocytes.

### I. Selection of 3-methyladenine DNA glycosylase for catalyzing transversion of adenine

1.1 Plasmid design and construction

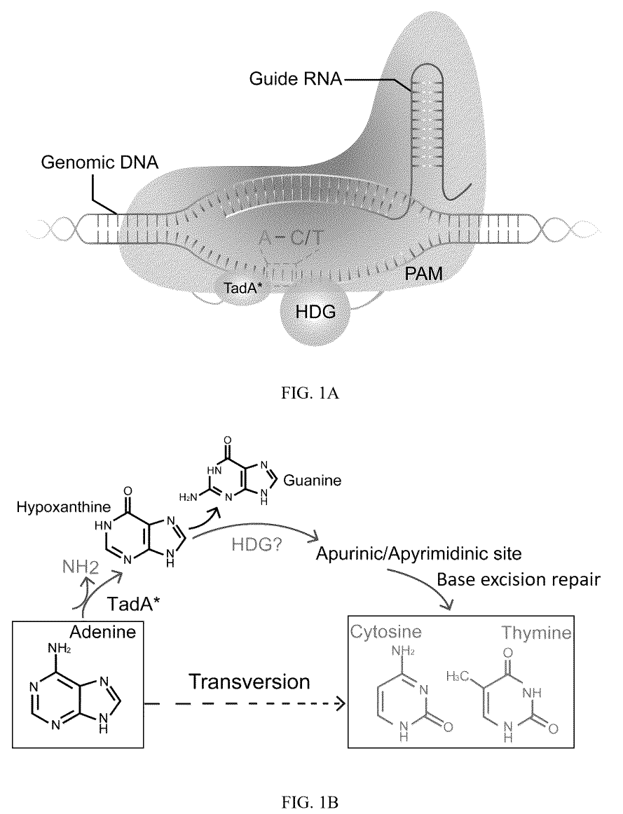

1.1.1 According to DNA base excising and repairing mechanism, we inferred that a deaminated product hypoxanthine (I) with adenine excised could achieve adenine transversion (FIGS. 1A-1B), under a combined action of Cas9 nuclease and adenosine deaminase, adenine of a target sequence in a genome was deaminated into hypoxanthine, the hypoxanthine was recognized/excised through 3-methyladenine DNA glycosylase, finally, an apurinic/apyrimidinic site was formed at this site, and in the end, A-to-C and A-to-T transversion occurred under the mediation of endogenous DNA damage repair.

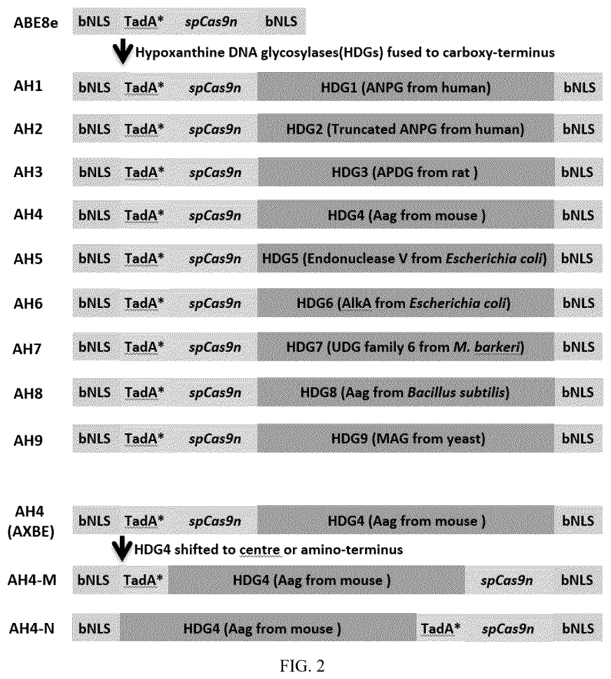

We obtained nine constructions by means of fusion design of 3-methyladenine DNA glycosylase (Aag) derived from different species (human, rats, mice, Bacillus subtilis and yeast) and other DNA glycosylases (HDGs) having a hypoxanthine recognizing/excising ability (endonuclease derived from E coli, and DNA glycosylase derived from Monascus barkeri) with Tad-8e derived from E coli and spcas9 nickase with impaired activity derived from Streptococcus pyogenes, and the 9 constructions were named as AH1, AH2, AH3, AH4, AH5, AH6, AH7, AH8 and AH9 respectively (FIG. 2). At the same time, two endogenous testing target sites PD-1-sg4 and PD-1-sg3 of a human gene (PD-1) and their sequences (Table 2) were designed for screening evaluation.

1.1.2 Nine HDGs sequences were synthesized according to gene sequences and amino acid sequences in Table 1, with ABE8e as a vector, and then seamless clonal assembly was performed. The target sites were as follows, two oligoes were synthesized according to Table 2, with CACC added to a forward strand and AAAC added to a reverse strand, and the annealed oligoes were linked to U6-sgRNA-EF1α-GFP that had been cleaved with BbsI enzyme.

1.1.3 Sanger sequencing of plasmids constructed in 1.1.1 and 1.1.2 was performed on to ensure complete accuracy.

TABLE 1
Gene sequences and amino acid sequences of HDGs used

| Name of | |
|---|---|
| sequence | Sequence (5′-3′) |
| HDG1 | Coding sequence (5′-3′): (SEQ ID NO: 1) |
| (ANPG | gtgacccccgccctgcagatgaagaagcccaagcagttctgcagaagaatgggccagaagaag |
| from | caaaggcccgccagagccggccaaccccatagcagctctgacgccgctcaggctcctgccgag |
| human) | caaccccacagctcgteggacgccgcccaggcacegtgtcccagagaaagatgcctgggcccc |
| | cccaccacccccggcccctacagaagcatctacttcagcagccccaagggccacctgaccagac |
| | tgggcctggagttcttcgaccagcccgccgtgcccctggccagagccttcctgggccaggtgctg |
| | gtgagaagactgcccaacggcaccgagctgagaggcagaatcgtggagaccgaggcctacctg |
| | ggccccgaagatgaggccgcccacagcagaggcggcagacagacccccagaaacagaggca |
| | tgttcatgaagcccggcaccctgtacgtgtacatcatctacggcatgtacttctgcatgaacatc |
| | agcagccagggcgacggcgcctgcgtgctgctgagagccctggagcccctggagggcctggagac |
| | catgagacagctgagaagcaccctgagaaagggcaccgccagcagagtgctgaaggacagag |
| | agctgtgcagcggccccagcaagctgtgccaggccctggccatcaacaagagcttcgaccagag |
| | agatctcgcgcaagatgaagcggtatggttagagagaggccccttagagccaagcgaacccgcc |
| | gtggtggcagccgccagagtgggtgttggccacgccggcgagtgggccagaaagcccctgaga |
| | ttctacgtgagaggcagcccctgggtgagcgtggtggacagagtggccgagcaggacacccag |
| | gcc |
| | Amino acid sequence: (SEQ ID NO: 5) |
| | VTPALQMKKPKQFCRRMGQKKQRPARAGQPHSSSDAAQAPA |
| | EQPHSSSDAAQAPCPRERCLGPPTTPGPYRSIYFSSPKGHLTRL |
| | GLEFFDQPAVPLARAFLGQVLVRRLPNGTELRGRIVETEAYLG |
| | PEDEAAHSRGGRQTPRNRGMFMKPGTLYVYIIYGMYFCMNIS |
| | SQGDGACVLLRALEPLEGLETMRQLRSTLRKGTASRVLKDRE |
| | LCSGPSKLCQALAINKSFDQRDLAQDEAVWLERGPLEPSEPAV |
| | VAAARVGVGHAGEWARKPLRFYVRGSPWVSVVDRVAEQDT |
| | QA |
| HDG2 | Coding sequence (5′-3′): (SEQ ID NO: 9) |
| (Truncated | agcaaggacagaagcatctacttcagcagccccaagggcctgctgaccagactgggcctggagt |
| ANPG from | tcttcgaccagcccgccgtgcccctggccagagccttcctgggccaggtgctggtgagaagactg |
| human) | cccaacggcaccgagctgagaggcagaatcgtggagaccgaggcctacctgggccccgaaga |
| | cgaggccgcccacagcagaggcggcagacagacccccagaaacagaggcatgttcatgaagc |
| | ccggcaccctgtacgtgtacatcatctacggcatgtacttctgcatgaacatcagcagccagggcg |
| | acggcgcctgcgtgctgctgagagccctggagcccctggagggcctggagaccatgagacacg |
| | tgagaagcaccctgagaaagggcaccgccagcagagtgctgaaggacagagagctgtgcagc |
| | ggccccagcaagctgtgccaggccctggccatcaacaagagcttcgaccagagagacctggctc |
| | aagacgaagctgtatggctggaaagaggcccgttggagccgagcgagcccgccgttgtagcag |
| | ccgcacgcgttggggtgggccacgccggcgagtgggccagaaagcccctgagattctacgtga |
| | gaggcagcccctgggtgagcgtggtggacagagtggccgagcaggacacccaggcc |
| | Amino acid sequence: (SEQ ID NO: 10) |
| | SKDRSIYFSSPKGLLTRLGLEFFDQPAVPLARAFLGQVLVRRLP |
| | NGTELRGRIVETEAYLGPEDEAAHSRGGRQTPRNRGMFMKPG |
| | TLYVYIIYGMYFCMNISSQGDGACVLLRALEPLEGLETMRHV |
| | RSTLRKGTASRVLKDRELCSGPSKLCQALAINKSFDQRDLAQD |
| | EAVWLERGPLEPSEPAVVAAARVGVGHAGEWARKPLRFYVR |
| | GSPWVSVVDRVAEQDTQA |
| HDG3 | Coding sequence (5′-3′): (SEQ ID NO: 2) |
| (ADPG | agaggccgtggcggcacggcaagactgggcagaggaagcctgaagcccgtaagcgtagtcctg |
| from rat) | cccgacaccgagcaccccgccttccccggcagaacacgaagacccggaaatgccagagccgg |
| | cagccaagtgaccggctctagagaggtgggccagatgcccgcccccctgagcagaaagatcgg |
| | ccagaagaagcagcagctggcccagagcgagcagcagcagacccccaaggagagactgagc |
| | agcacccccggcctgctgagaagcatctacttcagcagccccgaggacagacccgccagactgg |
| | ggcccgagtatttcgaccagcccgccgtgaccctggccagagccttcctgggccaggtgctggtg |
| | agaagactggccgacggcaccgagctgagaggcagaatcgtggagaccgaggcatatctgggc |
| | cccgaagatgaggcggctcacagcagagggggcaggcaaacccccagaaacagaggcatgtt |
| | catgaagcccggcaccctgtacgtgtacctgatctacggcatgtacttctgcctgaacgtatcct |
| | cccagggcgcaggtgcgtgtgtgctgctgagagccctggagcccctggagggcctggagaccatga |
| | gacagctgagaaacagcctgagaaagagcaccgtgggcagaagcctgaaggacagagagctgt |
| | gcaacggccccagcaagctgtgccaggccctggccatcgacaagagcttcgaccagagagactt |
| | agcccaggacgaggctgtgtggctggaacacgggcccctggaaagcagcagcccggcggtgg |
| | tggccgctgccagaatcggcatcggccacgccggcgagtggacccagaagcccctgagattcta |
| | cgtgcagggcagcccctgggtgagcgtcgtagacagagtggccgagcagatgtaccagcccca |
| | gcagaccgcctgcagcgactgcagcaaggtgaag |
| | Amino acid sequence: (SEQ ID NO: 6) |
| | RGRGGTARLGRGSLKPVSVVLPDTEHP AFPGRTRRPGNARAG |
| | SQVTGSREVGQMPAPLSRKIGQKKQQLAQSEQQQTPKERLSST |
| | PGLLRSIYFSSPEDRPARLGPEYFDQPAVTLARAFLGQVLVRRL |
| | ADGTELRGRIVETEAYLGPEDEAAHSRGGRQTPRNRGMFMKP |
| | GTLYVYLIYGMYFCLNVSSQGAGACVLLRALEPLEGLETMRQ |
| | LRNSLRKSTVGRSLKDRELCNGPSKLCQALAIDKSFDQRDLAQ |
| | DEAVWLEHGPLESSSPAVVAAARIGIGHAGEWTQKPLRFYVQ |
| | GSPWVSVVDRVAEQMYQPQQTACSDCSKVK |
| HDG4 | Coding sequence (5′-3′): (SEQ ID NO: 3) |
| (Aag from | ccggcgcggggcggctcagcccgtccagggagaggcgcactgaagcccgtgagcgtgaccct |
| mouse) | gctgcccgacaccgagcagccccccttcttaggcagagcgcgtagacctggcaatgctagagcg |
| | gggagcctggtgacaggataccacgaggtgggccagatgcccgcccccctgagcagaaagatc |
| | ggccagaagaagcagagactggccgatagcgagcagcagcagacccccaaggagagactgct |
| | gagcacccccggcctgagaagaagcatctacttcagcagccccgaggaccacagcggcagact |
| | gggcccagagtttttcgaccagcccgccgtgaccctggccagagccttcctgggccaggtgctgg |
| | tgagaagactggccgacggcaccgagctgagaggcagaatcgtggagaccgaggcctacttgg |
| | gacccgaggacgaggccgcccacagcagaggaggcagacagacccccagaaacagaggcatgtt |
| | catgaagcccggcaccctgtacgtgtacctgatctacggcatgtacttctgcttgaacgtgagct |
| | ctcagggcgccggcgcctgcgtactcctcagagccctggagcccctggagggcctggagaccat |
| | gagacagctgagaaacagcctgagaaagagcaccgtgggcagaagcctgaaggacagagagc |
| | tgtgcagcggccccagcaagctgtgccaggccctggccatcgacaagagcttcgaccagagaga |
| | cttggcgcaagatgacgccgtgtggctggaacacgggcccttggagagcagcagcccagccgta |
| | gtggtggcggccgccagaatcggcateggccacgccggcgagtggacccagaagcccctgag |
| | attctacgtgcagggcagcccctgggtgagcgtggtggacagagtggccgagcagatggacca |
| | gccccagcagaccgcctgcagcgagggcctgctgatcgtgcagaag |
| | Amino acid sequence: (SEQ ID NO: 7) |
| | PARGGSARPGRGALKPVSVTLLPDTEQPPFLGRARRPGNARA |
| | GSLVTGYHEVGQMP APLSRKIGQKKQRLADSEQQQTPKERLL |
| | STPGLRRSIYFSSPEDHSGRLGPEFFDQPAVTLARAFLGQVLVR |
| | RLADGTELRGRIVETEAYLGPEDEAAHSRGGRQTPRNRGMFM |
| | KPGTLYVYLIYGMYFCLNVSSQGAGACVLLRALEPLEGLETM |
| | RQLRNSLRKSTVGRSLKDRELCSGPSKLCQALAIDKSFDQRDL |
| | AQDDAVWLEHGPLESSSPAVVVAAARIGIGHAGEWTQKPLRF |
| | YVQGSPWVSVVDRVAEQMDQPQQTACSEGLLIVQK |
| HDG5 | Coding sequence (5′-3′): (SEQ ID NO: 11) |
| (endonucl | gacctggccagcctgagagcccagcagatcgagctggccagcagcgtgatcagagaggacag |
| ease V | gctggacaaggacccccccgacctgatcgccggggccgatgtgggttttgagcagggcggcga |
| from | ggtgaccagagccgccatggtgctgctgaagtaccccagcctggagctggtggagtacaaggtg |
| Escherichia | gccagaatcgccaccaccatgccctacatccccggcttcctgagcttcagagagtaccccgccctg |
| coli) | ctggccgcctgggagatgctgagccagaagcccgacctggtgttcgtggacggccacggcatca |
| | gccaccccagaagactgggcgtggccagccacttcggcctgctggtggacgtgcccaccatcgg |
| | cgtggccaagaagagattgtgtggcaagttcgaacccctatccagcgagcccggegccctggcc |
| | ccactgatggacaagggcgagcagctcgcctgggtgtggagaagcaaggccagatgcaacccc |
| | ctgttcatcgccaccggccacagagtgagcgtggacagcgccttagcctgggtgcagagatgcat |
| | gaagggctacagactgcccgagcccaccagatgggccgacgccgtggccagcgagagacccg |
| | ccttcgtgagatacaccgccaaccagccc |
| | Amino acid sequence: (SEQ ID NO: 12) |
| | DLASLRAQQIELASSVIREDRLDKDPPDLIAGADVGFEQGGEV |
| | TRAAMVLLKYPSLELVEYKVARIATTMPYIPGFLSFREYPALL |
| | AAWEMLSQKPDLVFVDGHGISHPRRLGVASHFGLLVDVPTIG |
| | VAKKRLCGKFEPLSSEPGALAPLMDKGEQLAWVWRSKARCN |
| | PLFIATGHRVSVDSALAWVQRCMKGYRLPEPTRWADAVASE |
| | RPAFVRYTANQP |
| HDG6 | Coding sequence (5′-3′): (SEQ ID NO: 13) |
| (AlkA | tacaccctgaactggcagcccccctacgactggagctggatgctgggcttcctggccgccagagc |
| from | cgtgagcggcgtggagaccgtggccgacagctactacgccagaagcctggccgtgggcgagta |
| Escherichia | cagaggcgtggtgaccgccatccccgacategccagacacaccctgcacatcaacctgagegcc |
| coli) | ggcctggagcccgtggccgccgagtgcctggccaagatgagcagactgttcgacctgcagtgta |
| | acccccagatagtgaacggggccctgggcaaactaggtgccgccagacccggtctgagactgcc |
| | cggctgtgtggacgccttcgagcagggcgtgagagccatcctgggccagctggtgagcgtggcc |
| | atggccgccaagctgaccagcagagtggcccagctgtacggcgagagactggacgacttcccc |
| | gactacgtatgctttcctaccccccagagattggcggtggccgacttgcaggccctgaaggccctg |
| | ggcatgcccctgaagcgtgcagaggccctgatccacctggccaatgccgcccttgaaggcacact |
| | gcctatgaccatccccggcgacgtggagcaggccatgaagaccctgcagaccttccccggcatc |
| | ggcagatggaccgccaactacttcgccctgagaggctggcaggccaaggacgtgttcctgcccg |
| | acgactacctgatcaagcagagattccccggcatgacccccgcccagatcagaagatacgccga |
| | gagatggaagccctggagaagctacgccctgctgcacatctggtacaccgagggctggcagccc |
| | gacgaggcc |
| | Amino acid sequence: (SEQ ID NO: 14) |
| | YTLNWQPPYDWSWMLGFLAARAVSGVETVADSYYARSLAV |
| | GEYRGVVTAIPDIARHTLHINLSAGLEPVAAECLAKMSRLFDL |
| | QCNPQIVNGALGKLGAARPGLRLPGCVDAFEQGVRAILGQLV |
| | SVAMAAKLTSRVAQLYGERLDDFPDYVCFPTPQRLAVADLQ |
| | ALKALGMPLKRAEALIHLANAALEGTLPMTIPGDVEQAMKTL |
| | QTFPGIGRWTANYFALRGWQAKDVFLPDDYLIKQRFPGMTPA |
| | QIRRYAERWKPWRSYALLHIWYTEGWQPDEA |
| HGD7 | Coding sequence (5′-3′): (SEQ ID NO: 15) |
| (UDG | aagaagcagggcttcccccccgtgatcgacgagaacaccgagatcctgatcctgggcagcctgc |
| family 6 | ccggcgacgtgagcatcagaaagcaccagtactacggccaccccggcaacgacttctggagact |
| from M. | gctgggcagcatcatcggcgaggacctgcagagcatcaactaccagaacagactggaggccctg |
| barkeri) | aagagaaacaagatcggcctgtgggacgtgttcaaggccggcaagagagagggcaacgagga |
| | caccaagatcaaggacgaggagatcaaccagttcagcatcctgaaggacatggcccccaacctg |
| | aagctggtgctgttcaacggcaagaagagcggcgagtacgagcccatcctgagagccatgggct |
| | acgagaccaagatcctgctgagcagcagcggcgccaacagaagaagcctgaagagcagaaag |
| | agcggctgggccgaggccttcaagaga |
| | Amino acid sequence: (SEQ ID NO: 16) |
| | KKQGFPPVIDENTEILILGSLPGDVSIRKHQYYGHPGNDFWRLL |
| | GSIIGEDLQSINYQNRLEALKRNKIGLWDVFKAGKREGNEDTK |
| | IKDEEINQFSILKDMAPNLKLVLFNGKKSGEYEPILRAMGYET |
| | KILLSSSGANRRSLKSRKSGWAEAFKR |
| HDG8 | Coding sequence (5′-3′): (SEQ ID NO: 4) |
| (Aag from | accagagagaagaaccccctgcccatcaccttctaccagaagaccgccctggagctggccccca |
| Bacillus | gcctgctgggctgcctgctggtgaaggagaccgacgagggcaccgccagcggctacatcgtgg |
| subtilis) | agaccgaggcctacatgggcgccggcgacagagccgcccacagcttcaacaacagaagaacc |
| | aagagaaccgagatcatgttcgccgaggccggcagagtgtacacctacgtgatgcacacccaca |
| | ccctgctgaacgtggtggccgccgaggaggacgtgccccaggccgtgctgatcagagccatcga |
| | gccccacgagggccagctgctgatggaggagagaagacccggcagaagccccagagagtgga |
| | ccaacggccccggcaagctgaccaaggccctgggcgtgaccatgaacgactacggcagatgga |
| | tcaccgagcagcccctgtacatcgagagcggctacacccccgaggccatcagcaccggcccca |
| | gaatcggcatcgacaacagcggcgaggccagagactacccctggagattctgggtgaccggca |
| | acagatacgtgagcaga |
| | Amino acid sequence: (SEQ ID NO: 8) |
| | TREKNPLPITFYQKTALELAPSLLGCLLVKETDEGTASGYIVET |
| | EAYMGAGDRAAHSFNNRRTKRTEIMFAEAGRVYTYVMHTHT |
| | LLNVVAAEEDVPQAVLIRAIEPHEGQLLMEERRPGRSPREWTN |
| | GPGKLTKALGVTMNDYGRWITEQPLYIESGYTPEAISTGPRIGI |
| | DNSGEARDYPWRFWVTGNRYVSR |
| HGD9 | Coding sequence (5′-3′): (SEQ ID NO: 17) |
| (MAG | aagctgaagagagagtacgacgagctgatcaaggccgacgccgtgaaggaaatcgccaaagaa |
| from | ctgggcagcagacccctggaggtggccctgccggagaaatatatcgccagacacgaggagaag |
| yeast) | ttcaacatggcctgcgagcacatcctggagaaggaccccagcctgttccccatcctgaagaacaa |
| | cgagttcaccctgtacctgaaggagacccaggtgcccaacaccctggaggactacttcatcaggct |
| | ggcaagcacgatattaagccagcagatcagcggccaggccgccgagagcatcaaggccagagt |
| | ggtgagcctgtacggcggcgccttccccgactacaagatcctgttcgaggacttcaaggaccccg |
| | ccaagtgcgccgaaatcgctaaatgtggtctgagcaagagaaagatgatctacctggagagcctg |
| | gccgtgtacttcaccgagaaatataaggacatcgagaagctgttcggccagaaggacaacgacga |
| | ggaggtgatcgagagcctggtgaccaacgtgaagggcatcggcccctggagcgccaagatgttc |
| | ctgatcagcggcctgaagagaatggacgtgttcgcccccgaggacctgggcatcgccagaggct |
| | tcagcaagtacctgagcgacaagcccgagctggagaaggagctgatgagagagagaaaggtgg |
| | tgaagaagagcaagatcaagcacaagaagtacaactggaagatctacgacgacgacatcatgga |
| | gaagtgcagcgagaccttcagcccctacagaagcgtgttcatgttcatcctgtggagactggccag |
| | caccaacacggacgccatgatgaaggccgaggagaacttcgtgaagagc |
| | Amino acid sequence: (SEQ ID NO: 18) |
| | KLKREYDELIKADAVKEIAKELGSRPLEVALPEKYIARHEEKF |
| | NMACEHILEKDPSLFPILKNNEFTLYLKETQVPNTLEDYFIRLA |
| | STILSQQISGQAAESIKARVVSLYGGAFPDYKILFEDFKDPAKC |
| | AEIAKCGLSKRKMIYLESLAVYFTEKYKDIEKLFGQKDNDEEV |
| | IESLVTNVKGIGPWSAKMFLISGLKRMDVFAPEDLGIARGFSK |
| | YLSDKPELEKELMRERKVVKKSKIKHKKYNWKIYDDDIMEKC |
| | SETFSPYRSVFMFILWRLASTNTDAMMKAEENFVKS |

TABLE 2
Target sites used and sequences

| Name of | |
|---|---|
| target site | Sequence (5′-3′) |
| PD-1-sg4 | CTTCCACATGAGCGTGGTCAGGG |
| | (SEQ ID NO: 19) |
| PD-1-sg3 | GGACCGCAGCCAGCCCGGCCAGG |
| | (SEQ ID NO: 20) |
| HBB 03 | CACGTTCACCTTGCCCCACAGGG |
| | (SEQ ID NO: 21) |
| EMX1-sg7 | GGCCCCAGTGGCTGCTCTGGGGG |
| | (SEQ ID NO: 22) |
| FANCF-M-b | AAGTTCGCTAATCCCGGAACTGG |
| | (SEQ ID NO: 23) |
| CCR5-sg1 | TAATAATTGATGTCATAGATTGG |
| | (SEQ ID NO: 24) |
| EMX1-sg1 | GCTCCCATCACATCAACCGGTGG |
| | (SEQ ID NO: 25) |
| FANCF site 2 | GCTGCAGAAGGGATTCCATGAGG |
| | (SEQ ID NO: 26) |
| CCR5-sg2 | GTGAGTAGAGCGGAGGCAGGAGG |
| | (SEQ ID NO: 27) |
| ABE site 27 | CGGGCATCAGAATTCCCTGGAGG |
| | (SEQ ID NO: 28) |
| HEK site 6 | CAAAGCAGGATGACAGGCAGGGG |
| | (SEQ ID NO: 29) |
| CCR5-sg5 | TTCAATGTAGACATCTATGTAGG |
| | (SEQ ID NO: 30) |
| hFGF6-sg2 | GCAGGTTAATGTTACAGCCCTGG |
| | (SEQ ID NO: 31) |

TABLE 3
Authentication primers of target sites used

| Name of target | |
|---|---|
| site | Sequence (5′-3′) |
| PD-1-sg4 | F: |
| | ggagtgagtacggtgtgcCGGAGAGCTTCGTGCTAAACTGGTA |
| | (SEQ ID NO: 32) |
| | R: gagttggatgctggatggCAGAGGTAGGTGCCGCTGTCATTG |
| | (SEQ ID NO: 33) |
| PD-1-sg3 | F: |
| | ggagtgagtacggtgtgcCAACACATCGGAGAGCTTCGTGCT |
| | A (SEQ ID NO: 34) |
| | R: |
| | gagttggatgctggatggTGACCACGCTCATGTGGAAGTCAC |
| | (SEQ ID NO: 35) |
| HBB 03 | F: ggagtgagtacggtgtgcAGCAACCTCAAACAGACACC |
| | (SEQ ID NO: 36) |
| | R: gagttggatgctggatggTGCCCAGTTTCTATTGGTCTCC |
| | (SEQ ID NO: 37) |
| EMX1-sg7 | F: ggagtgagtacggtgtgcATGGGAGCAGCTGGTCAGAGG |
| | (SEQ ID NO: 38) |
| | R: gagttggatgctggatggGGTTCTGGAACCACACCTTCAC |
| | (SEQ ID NO: 39) |
| FANCF-M-b | F: ggagtgagtacggtgtgcCTTTGGGCGGGGTCCAGTTCC |
| | (SEQ ID NO: 40) |
| | R: gagttggatgctggatggCTCTCTTGGAGTGTCTCCTCATC |
| | (SEQ ID NO: 41) |
| CCR5-sg1 | F: |
| | ggagtgagtacggtgtgcAAAACAGTTTGCATTCATGGAGGGC |
| | (SEQ ID NO: 42) |
| | R: |
| | gagttggatgctggatggTGAACACCAGTGAGTAGAGCGGAGG |
| | (SEQ ID NO: 43) |
| EMX1-sg1 | F: |
| | ggagtgagtacggtgtgcGTGGTTCCAGAACCGGAGGACAAA |
| | G (SEQ ID NO: 44) |
| | R: |
| | gagttggatgctggatggGTTTGTGGTTGCCCACCCTAGTCAT |
| | (SEQ ID NO: 45) |
| FANCF site 2 | F: ggagtgagtacggtgtgcGTAGCGCGCCCACTGCAAG (SEQ |
| | ID NO: 46) |
| | R: |
| | gagttggatgctggatggTTCCAATCAGTACGCAGAGAGTCGC |
| | (SEQ ID NO: 47) |
| CCR5-sg2 | F: ggagtgagtacggtgtgcTTTATTTATGCACAGGGTGGAAC |
| | (SEQ ID NO: 48) |
| | R: gagttggatgctggatggACCAGCATGTTGCCCACAA (SEQ |
| | ID NO: 49) |
| ABE site 27 | F: ggagtgagtacggtgtgcATCTCAGCGCTTTCGTCCAC (SEQ |
| | ID NO: 50) |
| | R: gagttggatgctggatggCTCATTTCCCCACTCCCTCC (SEQ |
| | ID NO: 51) |
| HEK site 6 | F: |
| | ggagtgagtacggtgtgcCCCTCCCTTCAAGATGGCTGACAAA |
| | (SEQ ID NO: 52) |
| | R: |
| | gagttggatgctggatggCCACTGTAGTCACACAGCACCAGAG |
| | (SEQ ID NO: 53) |
| CCR5-sg5 | F: ggagtgagtacggtgtgcCAGCAAACCTTCCCTTCACTAC |
| | (SEQ ID NO: 54) |
| | R: gagttggatgctggatggTCTTGTTCCACCCTGTGCATAA |
| | (SEQ ID NO: 55) |
| hFGF6-sg2 | F: ggagtgagtacggtgtgcCTGCTCACTTCATTCCTGCCTCAT |
| | (SEQ ID NO: 56) |
| | R: gagttggatgctggatggCCATCATCGCCCTGACGTCAACC |
| | (SEQ ID NO: 57) |

### 1.2 Cell Transfection

Day 1 293T Cells were Planted on a 24-Well Plate;

(1) HEK293T cells were digested, and inoculated on a 24-well plate according to 2×10⁵ cells/well.

Note: after cell thawing, it is generally necessary to passage 2 times before the cells can be used for transfection experiments.

### Day 2 Transfection

(2) States of the cells in each well were observed.

Note: it is required that the cell density before transfection should be 70%-90%, and the states should be normal.

(3) A plasmid transfection amount was as follows, with ABE8e as the control.

Newly constructed plasmids in 1.1: U6-sgRNA-EF1α-GFP=750 ng: 250 ng

Each group was set to three biologically replicates (n=3).

### 1.3 Genome Extraction and Preparation of Amplicon Library

Cell genome DNA was extracted by using a Tiangen cell genome extraction kit (DP304) after 72 h transfection. Afterwards, corresponding site-specific primers (see Table 3) were designed. By using an operating process of a Hitom kit, that is, a bridging sequence 5′-ggagtgagtacggtgtgc-3′ (SEQ ID NO: 58) was added to the 5′ terminus of a forward site-specific primer, and a bridging sequence 5′-gagttggatgctggatgg-3′ (SEQ ID NO: 59) was added to the 5′ terminus of a reverse site-specific primer. Genome loci of interest were amplified with primers to obtain a first-round PCR product, then a second-round PCR product was obtained by using the first-round PCR product as a template, and then the products were mixed together for gel-cutting recovery and purification and then sent to the company for Illumina sequencing.

### 1.4 Deep Sequencing Result Analysis and Statistics

Deep sequencing results were analyzed by using the BE-analyzer website, that is, the editing efficiency of A-to-C, A-to-T and A-to-G was calculated, and statistical plotting was performed by using graphpad prism 9.1.0.

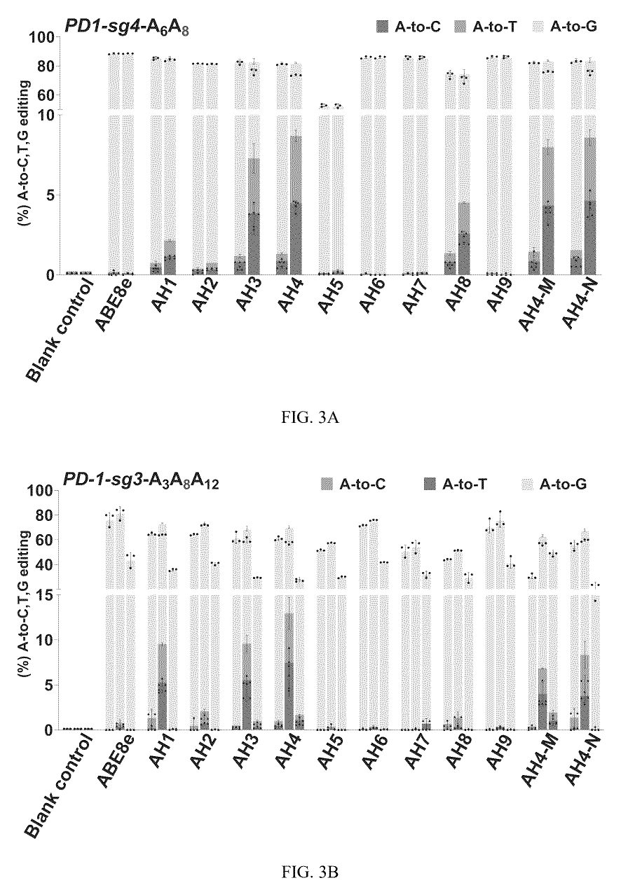

It was found according to the deep sequencing results that, only 3-methyladenine DNA glycosylase derived from mice, rats and human and Aag derived from Bacillus subtilis had the ability of mutating A-to-C and A-to-T, a control group ABE8e was unable to produce A-based transversion, while a construction AH4 fused with Aag derived from mice exhibited the optimal transversion ability, the A-to-C and A-to-T efficiency at the target site PD-1-sg4 was 4.5% and 4.3%, respectively, and the A-to-C and A-to-T efficiency at the target site PD-1-sg3 was 7.4% and 5.5%, respectively (FIGS. 3A-3B).

### II. Comparison of Adenine Editing Conditions Produced by AH4, AH4-M and AH4-N

### 2.1 Plasmid Design and Construction

2.1.1 The above experiments were all carried out by fusing Aag at the C terminus. In order to further study the influence of placing the Aag derived from mice at different positions on the production of A-to-C and A-to-T, the Aag was fused at the middle terminus and the N terminus, and an AH4-M construction and an AH4-N construction (Table 2) were obtained via seamless clonal assembly. At the same time, five endogenous target sites HBB 03, EMX1-sg7, FANCF-M-b, CCR5-sg1 and EMX1-sg1 from human were designed for testing (Table 2), and the construction method was the same as 1.1.2.

2.1.2 Sanger sequencing was performed on plasmids constructed in 2.1.1 to ensure complete accuracy.

### 2.2 Cell Transfection

Day 1 293T Cells were Planted on a 24-Well Plate;

(1) HEK293T cells were digested, and inoculated on a 24-well plate according to 2×10⁵ cells/well.

Note: after cell thawing, it is generally necessary to passage 2 times before the cells can be used for transfection experiments.

### Day 2 Transfection

(2) States of the cells in each well were observed.

Note: it is required that the cell density before transfection shall be 70%-90%, and the states shall be normal.

(3) A plasmid transfection amount was as follows, with ABE8e as the control;

Newly constructed plasmids in 2.1: U6-sgRNA-EF1α-GFP=750 ng: 250 ng

Each group was set to three biologically replicates (n=3).

### 2.3 Genome Extraction and Preparation of Amplicon Library

Cell genome DNA was extracted by using a Tiangen cell genome extraction kit (DP304) after 72 h transfection. Afterwards, corresponding site-specific primers (see Table 3) were designed. By using an operating process of a Hitom kit, a bridging sequence 5′-ggagtgagtacggtgtgc-3′ (SEQ ID NO: 58) was added to the 5′ terminus of a forward site-specific primer, and a bridging sequence 5′-gagttggatgctggatgg-3′ (SEQ ID NO: 59) was added to the 5′ terminus of a reverse site-specific primer. Genome loci of interest were amplified with primers to obtain a first-round PCR product, then a second-round PCR product was obtained by using the first-round PCR product as a template, and then the products were mixed together for gel-cutting recovery and purification and then sent to the company for Illumina sequencing.

### 2.4 Deep Sequencing Result Analysis and Statistics

Deep sequencing results were analyzed by using the BE-analyzer website, that is, the editing efficiency of A-to-C, A-to-T and A-to-G was calculated, and statistical plotting was performed by using graphpad prism 9.1.0.

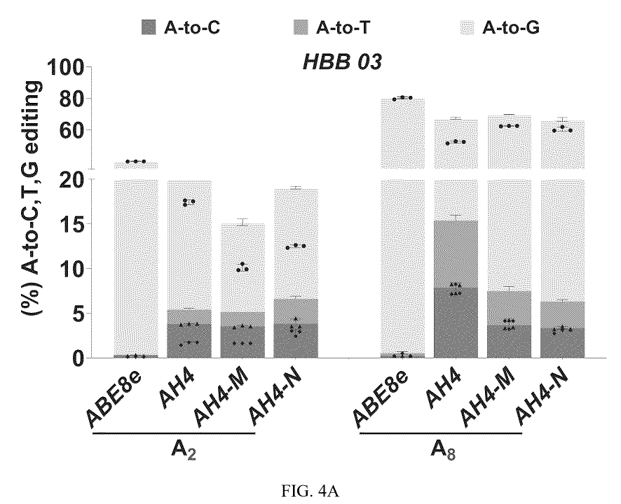

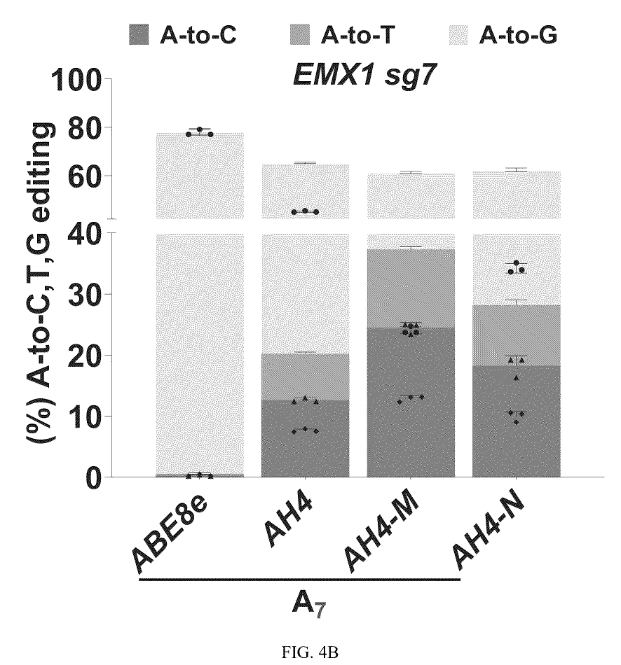

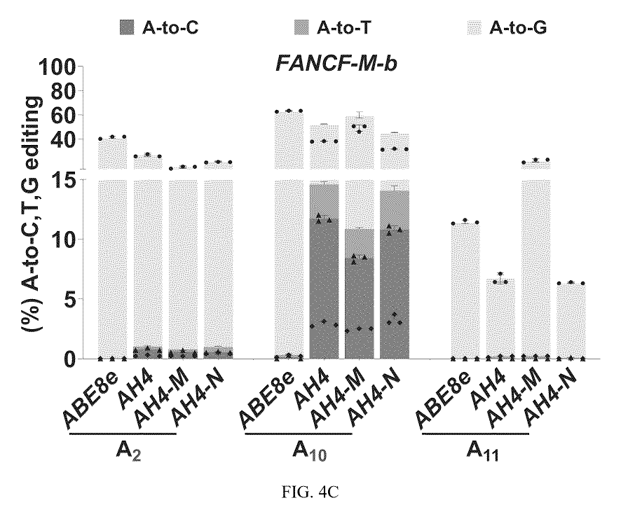

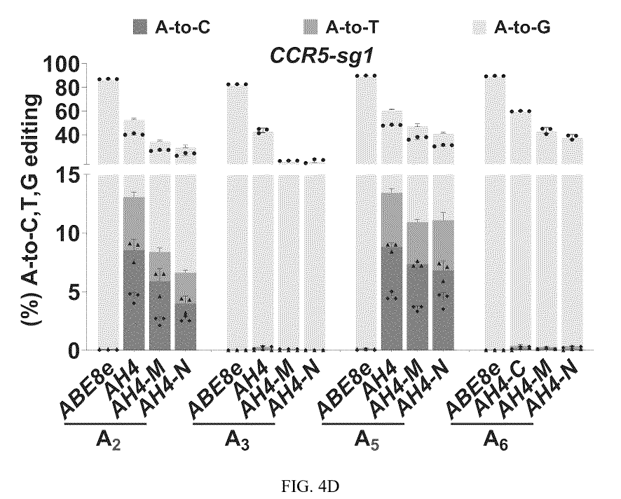

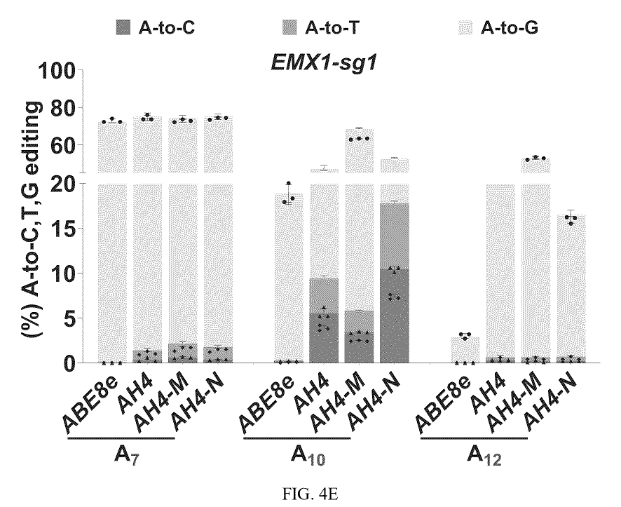

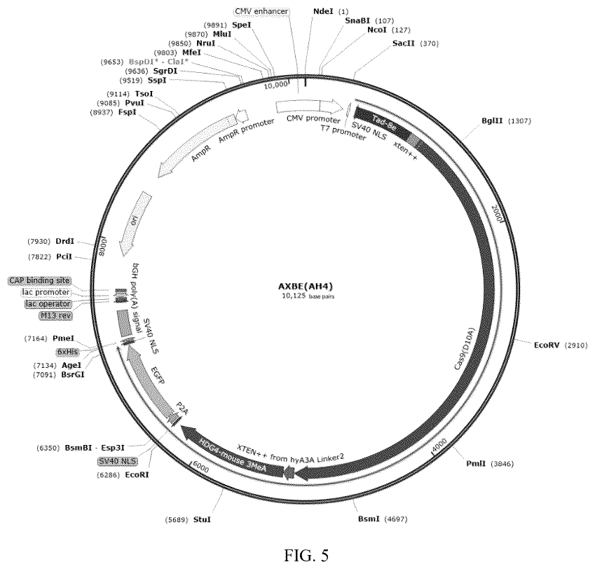

In this experiment, the target site PD-1-sg4 and the target site PD-1-sg3 were also used for evaluation, the A-to-C efficiency of the AH4-M and the AH4-N was 4.3% and 4.6%, respectively, the A-to-T efficiency was 3.6% and 3.9% respectively, and the transversion of A produced by the AH4-M and the AH4-N at the two target sites was lower than that of the AH4 (FIGS. 3A-3B). In order to evaluate the ability of executing transversion editing on adenine by the Aag at different positions more objectively and fairly, another five endogenous target sites were further designed for secondary validation, and the results showed that (FIGS. 4A-4E): a control group ABE8e was unable to produce mutations from A to C and from A to T at the five target sites, for the AH4, it exhibited the optimal transversion effect at the three endogenous target sites HBB 03, FANCF-M-b and CCR5-sg1, the highest editing efficiency of A-to-C at the three target sites was 7.8%, 11.7% and 8.8% respectively, the highest editing efficiency of A-to-T at the three target sites was 7.5%, 2.9% and 4.6% respectively, but at particular target sites, the AH4-M or the AH4-N was the optimal in exhibition, for example, at the target site EMX1-sg7, the editing efficiency of A-to-C caused by the AH4-M reached 24.4% and the editing efficiency of catalyzing A-to-T reached 12.8%, for the target site EMX1-sg1 and the target site HBG-sg1, the editing efficiency of A-to-C caused by the AH4-N could reach 10.4% and the editing efficiency of catalyzing A-to-T reached 7.3%, in general, the Aag had certain editing efficiency no matter whether it is fused at the C terminus or the middle terminus or the N terminus, in an actual fusing process, different fusion terminuses could be selected for different target sites, in conjunction with the editing conditions of the above seven target sites, and we selected the more stable AH4 as a final base editor which was named as AXBE (composed of CMV-TadA8e-Cas9 nickase-HDG4-BGH polyA, where a constructed plasmid profile was as shown in FIG. 5), which could achieve A⋅T to C⋅G and A⋅T to T⋅A in mammal cells.

### III. Validation of Editing Characteristics of AXBE

### 3.1 Plasmid Design and Construction

3.1.1 In order to further evaluate editing characteristics of the AXBE, six endogenous testing target sites FANCF site 2, CCR5-sg2, ABE site 27, HEK site 6, CCR5-sg5 and hFGF6-sg2 (Table 2) were further designed, with the ABE8e as the control.

3.1.2 Sanger sequencing was performed on plasmids constructed in 3.1.1 to ensure complete accuracy.

### 3.2 Cell Transfection

Day 1 293T Cells were Planted on a 24-Well Plate

(1) HEK293T cells were digested, and inoculated on a 24-well plate according to 2×10⁵ cells/well.

Note: after cell thawing, it is generally necessary to passage 2 times before the cells can be used for transfection experiments.

### Day 2 Transfection

(2) States of the cells in each well were observed.

Note: it is required that the cell density before transfection shall be 70%-90%, and the states shall be normal.

(3) A plasmid transfection amount was as follows, with ABE8e as the control

Newly constructed plasmids in 3.1: U6-sgRNA-EF1α-GFP=750 ng: 250 ng

Each group was set to three biologically replicates (n=3).

### 3.3 Genome Extraction and Preparation of Amplicon Library

Cell genome DNA was extracted by using a Tiangen cell genome extraction kit (DP304) after 72 h transfection. Afterwards, corresponding site-specific primers (see Table 3) were designed. By using an operating process of a Hitom kit, a bridging sequence 5′-ggagtgagtacggtgtgc-3′ (SEQ ID NO: 58) was added to the 5′ terminus of a forward site-specific primer, and a bridging sequence 5′-gagttggatgctggatgg-3′ (SEQ ID NO: 59) was added to the 5′ terminus of a reverse site-specific primer. Genome loci of interest were amplified with primers to obtain a first-round PCR product, then a second-round PCR product was obtained by using the first-round PCR product as a template, and then the products were mixed together for gel-cutting recovery and purification and then sent to the company for Illumina sequencing.

### 3.4 Deep Sequencing Result Analysis and Statistics

Deep sequencing results were analyzed by using the BE-analyzer website, that is, the editing efficiency of A-to-C, A-to-T and A-to-G was calculated, and statistical plotting was performed by using graphpad prism 9.1.0.

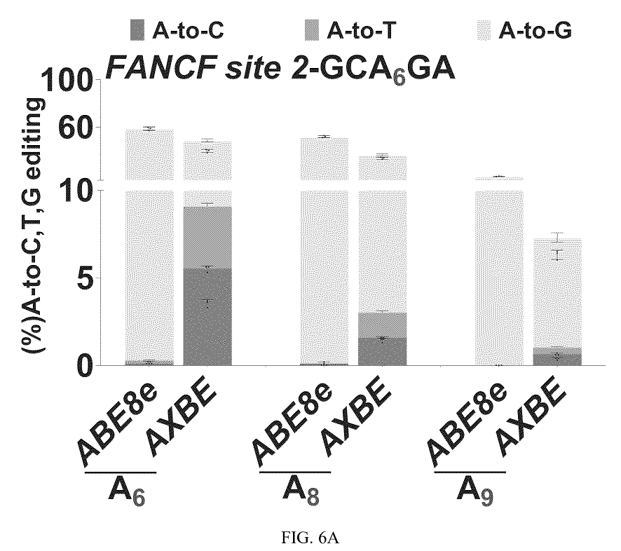

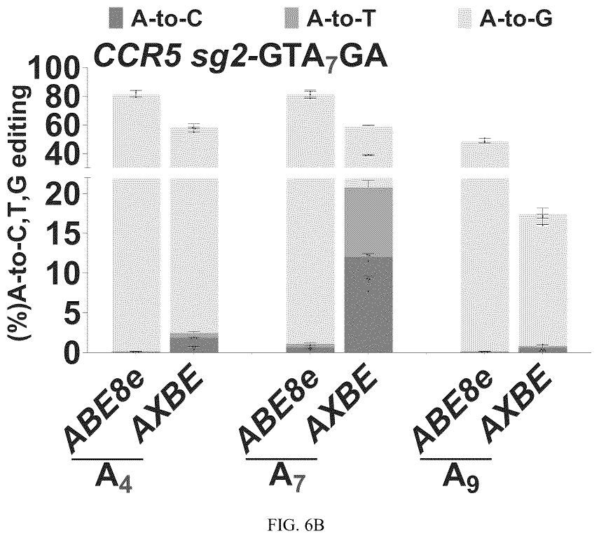

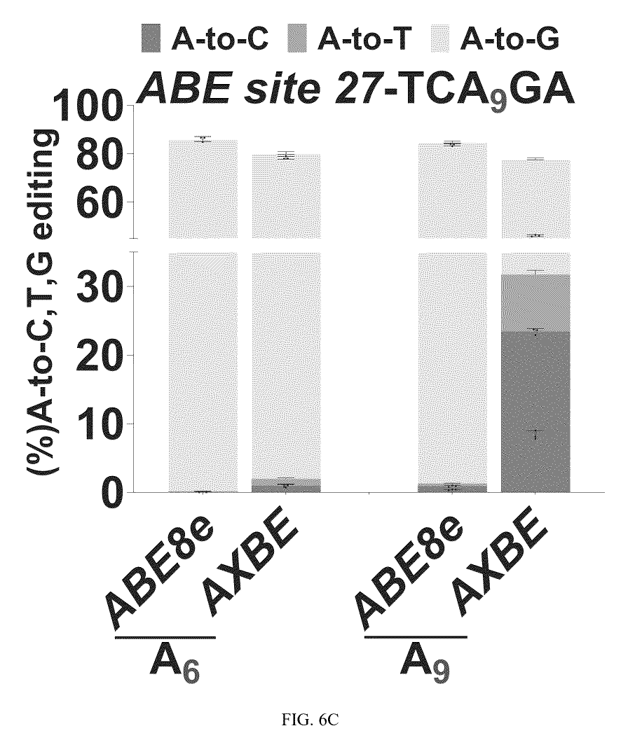

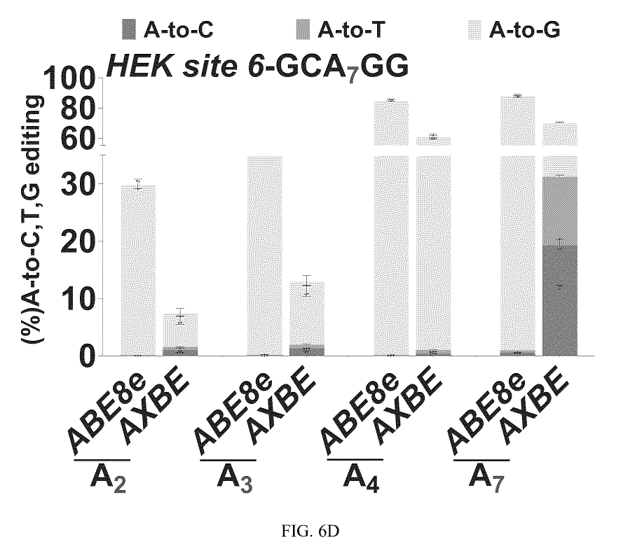

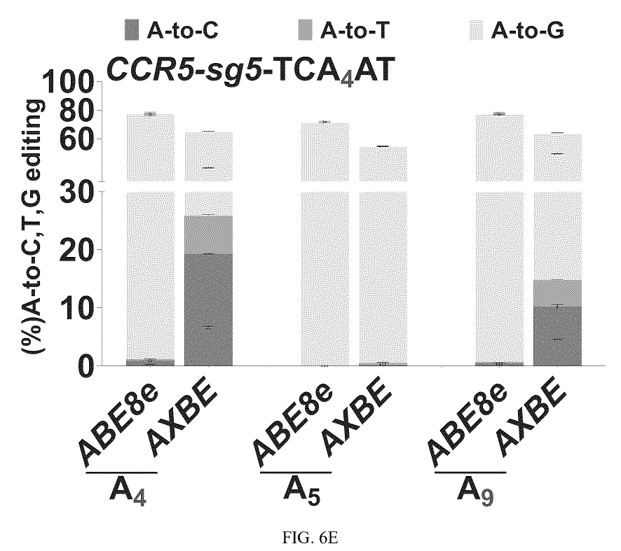

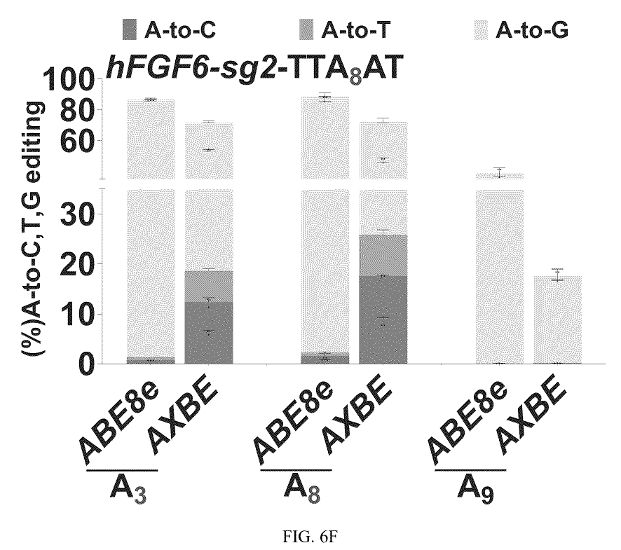

The results showed that (FIGS. 6A-6F): the editing efficiency of A-to-C of the AXBE at the six target sites (highest value at each target site) was 5.5%-23.4%, the average editing efficiency of A-to-C at the six target sites was 15.3%, the editing efficiency of A-to-T at the six target sites (a highest value was taken at each target site) was 3.5%-12%, the average editing efficiency of A-to-T at the six target sites was 7.6%, and in conjunction with the seven endogenous target sites tested previously, it was found according to the editing characteristics of all the 13 target sites that an editing window range of A-to-C and A-to-T was mainly located at A2-A10 (NGG was recorded as 21-23). To sum up, the AXBE could effectively mediate adenine-based transversion with mammal cells, and was expected to treat SNP related to 16% of C⋅G to A⋅T diseases or SNP related to 7% of T⋅A to A⋅T diseases, which would also greatly promote the use in human disease model production, and crop genetic breeding, etc.

The above embodiments are only preferred embodiments of the present disclosure and are only intended to explain the present disclosure, not to limit the implementation scope of the present disclosure. For those skilled in the art, other implementations can be easily made by substitution or modification based on the technical content disclosed in this specification. Therefore, any changes or improvements made to the principles of the present disclosure, etc., all should be included within the scope of the patent application for the present disclosure.

What is claimed is:

1. A base editing system for achieving A-to-C and/or A-to-T base mutations, comprising adenosine deaminase TadA, Cas9 nuclease, 3-methyladenine DNA glycosylase, and variants of the 3-methyladenine DNA glycosylase.

2. The base editing system for achieving the A-to-C and/or A-to-T base mutations according to claim 1, wherein the gene sequence of the 3-methyladenine DNA glycosylase is as shown in one of SEQ ID NOS: 2-4.

3. The base editing system for achieving the A-to-C and/or A-to-T base mutations according to claim 1, wherein the amino acid sequence of the 3-methyladenine DNA glycosylase is as shown in one of SEQ ID NOS: 6-8.

4. The base editing system for achieving the A-to-C and/or A-to-T base mutations according to claim 1, wherein the 3-methyladenine DNA glycosylase is derived from rats, mice, or Bacillus subtilis.

5. The base editing system for achieving the A-to-C and/or A-to-T base mutations according to claim 1, wherein sources of the adenosine deaminase TadA comprise E. coli, Staphylococcus aureus, Oceanobacillus sojae, and Acinetobacter; and
the Cas9 nuclease comprises spCas9 derived from Saccharomyces cerevisiae, Cas9 nickase, and variants VQR-spCas9, VRER-spCas9, spRY, and spNG thereof, SaCas9 derived from Staphylococcus aureus and variants SaCas9-KKH and SaCas9-NG thereof, LbCas12a derived from Lachnospiraceae, and enAsCas12a derived from Acidaminococcus, the Cas9 nuclease is further configured to be replaced with nucleases to capable of specifically recognizing DNA and having a cutting function, the Cas9 nickase is derived from Streptococcus pyogenes.

6. A base editing method for achieving A-to-C and/or A-to-T base mutations, comprising the following step:
expressing the adenosine deaminase TadA, the Cas9 nuclease, and the 3-methyladenine DNA glycosylase according to claim 1 in a host to perform base editing on a target gene in a genome of the host, wherein-preferably, the host is eukaryotic cells.

7. The base editing method for achieving the A-to-C and/or A-to-T base mutations according to claim 6, wherein the “expressing the adenosine deaminase TadA, the Cas9 nuclease, and the 3-methyladenine DNA glycosylase in the host” is expressing a coding gene of the adenosine deaminase TadA, the Cas9 nuclease, and the 3-methyladenine DNA glycosylase by introducing the coding gene of the adenosine deaminase TadA, the Cas9 nuclease, and the 3-methyladenine DNA glycosylase into the eukaryotic cells to achieve the A-to-C and/or A-to-T base mutations.

8. The base editing method for achieving the A-to-C and/or A-to-T base mutations according to claim 6, wherein a specific achieving process of the A-to-C and/or A-to-T base mutations is: under a combined action of the Cas9 nuclease and the adenosine deaminase TadA, adenine of a target sequence in the genome is deaminated into hypoxanthine, the hypoxanthine is recognized/excised through the 3-methyladenine DNA glycosylase, an apurinic/apyrimidinic site is formed at a site of the adenine, and a transversion of A-to-C and/or A-to-T occurs under a mediation of endogenous DNA damage repair, wherein an editing window range of the target gene is A2-A10.

9. A product comprising the base editing system according to claim 1, wherein the product comprises a kit and a pharmaceutical composition.

10. A method of achieving A-to-C and/or A-to-T base mutations, comprising using the product according to claim 9.

11. The base editing system for achieving the A-to-C and/or A-to-T base mutations according to claim 5, wherein the adenosine deaminase TadA is derived from the E. coli.

12. The base editing system for achieving the A-to-C and/or A-to-T base mutations according to claim 11, wherein the adenosine deaminase TadA derived from the E. coli is TadA-8e.

13. The base editing method for achieving the A-to-C and/or A-to-T base mutations according to claim 6, wherein the host is mammalian cells.

14. The base editing method for achieving the A-to-C and/or A-to-T base mutations according to claim 13, wherein the host is cells derived from rats, mice, or Bacillus subtilis.

15. A base editing method for achieving A-to-C and/or A-to-T base mutations, comprising the following step:
expressing the adenosine deaminase TadA, the Cas9 nuclease, and the 3-methyladenine DNA glycosylase according to claim 2 in a host to perform base editing on a target gene in a genome of the host, wherein the host is eukaryotic cells.

16. A base editing method for achieving A-to-C and/or A-to-T base mutations, comprising the following step:
expressing the adenosine deaminase TadA, the Cas9 nuclease, and the 3-methyladenine DNA glycosylase according to claim 3 in a host to perform base editing on a target gene in a genome of the host, wherein the host is eukaryotic cells.

17. A base editing method for achieving A-to-C and/or A-to-T base mutations, comprising the following step:
expressing the adenosine deaminase TadA, the Cas9 nuclease, and the 3-methyladenine DNA glycosylase according to claim 4 in a host to perform base editing on a target gene in a genome of the host, wherein the host is eukaryotic cells.

18. A base editing method for achieving A-to-C and/or A-to-T base mutations, comprising the following step:
expressing the adenosine deaminase TadA, the Cas9 nuclease, and the 3-methyladenine DNA glycosylase according to claim 5 in a host to perform base editing on a target gene in a genome of the host, wherein the host is eukaryotic cells.

19. The base editing method for achieving the A-to-C and/or A-to-T base mutations according to claim 15, wherein the host is mammalian cells.

20. The base editing method for achieving the A-to-C and/or A-to-T base mutations according to claim 19, wherein the host is cells derived from rats, mice, or Bacillus subtilis.


SEQUENCE LISTING (59 sequences)

SEQ ID NO: 1 - DNA, 891 bp, Homo sapiens
gtgacccccgccctgcagatgaagaagcccaagcagttctgcagaagaatgggccagaagaagcaaaggcccgccagagccggccaaccccatagcagctctgacgccgctcaggctcctgccgagcaaccccacagctcgtcggacgccgcccaggcaccgtgtcccagagaaagatgcctgggcccccccaccacccccggcccctacagaagcatctacttcagcagccccaagggccacctgaccagactgggcctggagttcttcgaccagcccgccgtgcccctggccagagccttcctgggccaggtgctggtgagaagactgcccaacggcaccgagctgagaggcagaatcgtggagaccgaggcctacctgggccccgaagatgaggccgcccacagcagaggcggcagacagacccccagaaacagaggcatgttcatgaagcccggcaccctgtacgtgtacatcatctacggcatgtacttctgcatgaacatcagcagccagggcgacggcgcctgcgtgctgctgagagccctggagcccctggagggcctggagaccatgagacagctgagaagcaccctgagaaagggcaccgccagcagagtgctgaaggacagagagctgtgcagcggccccagcaagctgtgccaggccctggccatcaacaagagcttcgaccagagagatctcgcgcaagatgaagcggtatggttagagagaggccccttagagccaagcgaacccgccgtggtggcagccgccagagtgggtgttggccacgccggcgagtgggccagaaagcccctgagattctacgtgagaggcagcccctgggtgagcgtggtggacagagtggccgagcaggacacccaggcc

SEQ ID NO: 2 - DNA, 984 bp, Rattus norvegicus
agaggccgtggcggcacggcaagactgggcagaggaagcctgaagcccgtaagcgtagtcctgcccgacaccgagcaccccgccttccccggcagaacacgaagacccggaaatgccagagccggcagccaagtgaccggctctagagaggtgggccagatgcccgcccccctgagcagaaagatcggccagaagaagcagcagctggcccagagcgagcagcagcagacccccaaggagagactgagcagcacccccggcctgctgagaagcatctacttcagcagccccgaggacagacccgccagactggggcccgagtatttcgaccagcccgccgtgaccctggccagagccttcctgggccaggtgctggtgagaagactggccgacggcaccgagctgagaggcagaatcgtggagaccgaggcatatctgggccccgaagatgaggcggctcacagcagagggggcaggcaaacccccagaaacagaggcatgttcatgaagcccggcaccctgtacgtgtacctgatctacggcatgtacttctgcctgaacgtatcctcccagggcgcaggtgcgtgtgtgctgctgagagccctggagcccctggagggcctggagaccatgagacagctgagaaacagcctgagaaagagcaccgtgggcagaagcctgaaggacagagagctgtgcaacggccccagcaagctgtgccaggccctggccatcgacaagagcttcgaccagagagacttagcccaggacgaggctgtgtggctggaacacgggcccctggaaagcagcagcccggcggtggtggccgctgccagaatcggcatcggccacgccggcgagtggacccagaagcccctgagattctacgtgcagggcagcccctgggtgagcgtcgtagacagagtggccgagcagatgtaccagccccagcagaccgcctgcagcgactgcagcaaggtgaag

SEQ ID NO: 3 - DNA, 996 bp, Mus musculus
ccggcgcggggcggctcagcccgtccagggagaggcgcactgaagcccgtgagcgtgaccctgctgcccgacaccgagcagccccccttcttaggcagagcgcgtagacctggcaatgctagagcggggagcctggtgacaggataccacgaggtgggccagatgcccgcccccctgagcagaaagatcggccagaagaagcagagactggccgatagcgagcagcagcagacccccaaggagagactgctgagcacccccggcctgagaagaagcatctacttcagcagccccgaggaccacagcggcagactgggcccagagtttttcgaccagcccgccgtgaccctggccagagccttcctgggccaggtgctggtgagaagactggccgacggcaccgagctgagaggcagaatcgtggagaccgaggcctacttgggacccgaggacgaggccgcccacagcagaggaggcagacagacccccagaaacagaggcatgttcatgaagcccggcaccctgtacgtgtacctgatctacggcatgtacttctgcttgaacgtgagctctcagggcgccggcgcctgcgtactcctcagagccctggagcccctggagggcctggagaccatgagacagctgagaaacagcctgagaaagagcaccgtgggcagaagcctgaaggacagagagctgtgcagcggccccagcaagctgtgccaggccctggccatcgacaagagcttcgaccagagagacttggcgcaagatgacgccgtgtggctggaacacgggcccttggagagcagcagcccagccgtagtggtggcggccgccagaatcggcatcggccacgccggcgagtggacccagaagcccctgagattctacgtgcagggcagcccctgggtgagcgtggtggacagagtggccgagcagatggaccagccccagcagaccgcctgcagcgagggcctgctgatcgtgcagaag

SEQ ID NO: 4 - DNA, 585 bp, Bacillus subtilis
accagagagaagaaccccctgcccatcaccttctaccagaagaccgccctggagctggcccccagcctgctgggctgcctgctggtgaaggagaccgacgagggcaccgccagcggctacatcgtggagaccgaggcctacatgggcgccggcgacagagccgcccacagcttcaacaacagaagaaccaagagaaccgagatcatgttcgccgaggccggcagagtgtacacctacgtgatgcacacccacaccctgctgaacgtggtggccgccgaggaggacgtgccccaggccgtgctgatcagagccatcgagccccacgagggccagctgctgatggaggagagaagacccggcagaagccccagagagtggaccaacggccccggcaagctgaccaaggccctgggcgtgaccatgaacgactacggcagatggatcaccgagcagcccctgtacatcgagagcggctacacccccgaggccatcagcaccggccccagaatcggcatcgacaacagcggcgaggccagagactacccctggagattctgggtgaccggcaacagatacgtgagcaga

SEQ ID NO: 5 - protein, 297 aa, Homo sapiens
VTPALQMKKPKQFCRRMGQKKQRPARAGQPHSSSDAAQAPAEQPHSSSDAAQAPCPRERCLGPPTTPGPYRSIYFSSPKGHLTRLGLEFFDQPAVPLARAFLGQVLVRRLPNGTELRGRIVETEAYLGPEDEAAHSRGGRQTPRNRGMFMKPGTLYVYIIYGMYFCMNISSQGDGACVLLRALEPLEGLETMRQLRSTLRKGTASRVLKDRELCSGPSKLCQALAINKSFDQRDLAQDEAVWLERGPLEPSEPAVVAAARVGVGHAGEWARKPLRFYVRGSPWVSVVDRVAEQDTQA

SEQ ID NO: 6 - protein, 328 aa, Rattus norvegicus
RGRGGTARLGRGSLKPVSVVLPDTEHPAFPGRTRRPGNARAGSQVTGSREVGQMPAPLSRKIGQKKQQLAQSEQQQTPKERLSSTPGLLRSIYFSSPEDRPARLGPEYFDQPAVTLARAFLGQVLVRRLADGTELRGRIVETEAYLGPEDEAAHSRGGRQTPRNRGMFMKPGTLYVYLIYGMYFCLNVSSQGAGACVLLRALEPLEGLETMRQLRNSLRKSTVGRSLKDRELCNGPSKLCQALAIDKSFDQRDLAQDEAVWLEHGPLESSSPAVVAAARIGIGHAGEWTQKPLRFYVQGSPWVSVVDRVAEQMYQPQQTACSDCSKVK

SEQ ID NO: 7 - protein, 332 aa, Mus musculus
PARGGSARPGRGALKPVSVTLLPDTEQPPFLGRARRPGNARAGSLVTGYHEVGQMPAPLSRKIGQKKQRLADSEQQQTPKERLLSTPGLRRSIYFSSPEDHSGRLGPEFFDQPAVTLARAFLGQVLVRRLADGTELRGRIVETEAYLGPEDEAAHSRGGRQTPRNRGMFMKPGTLYVYLIYGMYFCLNVSSQGAGACVLLRALEPLEGLETMRQLRNSLRKSTVGRSLKDRELCSGPSKLCQALAIDKSFDQRDLAQDDAVWLEHGPLESSSPAVVVAAARIGIGHAGEWTQKPLRFYVQGSPWVSVVDRVAEQMDQPQQTACSEGLLIVQK

SEQ ID NO: 8 - protein, 195 aa, Bacillus subtilis
TREKNPLPITFYQKTALELAPSLLGCLLVKETDEGTASGYIVETEAYMGAGDRAAHSFNNRRTKRTEIMFAEAGRVYTYVMHTHTLLNVVAAEEDVPQAVLIRAIEPHEGQLLMEERRPGRSPREWTNGPGKLTKALGVTMNDYGRWITEQPLYIESGYTPEAISTGPRIGIDNSGEARDYPWRFWVTGNRYVSR

SEQ ID NO: 9 - DNA, 690 bp, Homo sapiens
agcaaggacagaagcatctacttcagcagccccaagggcctgctgaccagactgggcctggagttcttcgaccagcccgccgtgcccctggccagagccttcctgggccaggtgctggtgagaagactgcccaacggcaccgagctgagaggcagaatcgtggagaccgaggcctacctgggccccgaagacgaggccgcccacagcagaggcggcagacagacccccagaaacagaggcatgttcatgaagcccggcaccctgtacgtgtacatcatctacggcatgtacttctgcatgaacatcagcagccagggcgacggcgcctgcgtgctgctgagagccctggagcccctggagggcctggagaccatgagacacgtgagaagcaccctgagaaagggcaccgccagcagagtgctgaaggacagagagctgtgcagcggccccagcaagctgtgccaggccctggccatcaacaagagcttcgaccagagagacctggctcaagacgaagctgtatggctggaaagaggcccgttggagccgagcgagcccgccgttgtagcagccgcacgcgttggggtgggccacgccggcgagtgggccagaaagcccctgagattctacgtgagaggcagcccctgggtgagcgtggtggacagagtggccgagcaggacacccaggcc

SEQ ID NO: 10 - protein, 230 aa, Homo sapiens
SKDRSIYFSSPKGLLTRLGLEFFDQPAVPLARAFLGQVLVRRLPNGTELRGRIVETEAYLGPEDEAAHSRGGRQTPRNRGMFMKPGTLYVYIIYGMYFCMNISSQGDGACVLLRALEPLEGLETMRHVRSTLRKGTASRVLKDRELCSGPSKLCQALAINKSFDQRDLAQDEAVWLERGPLEPSEPAVVAAARVGVGHAGEWARKPLRFYVRGSPWVSVVDRVAEQDTQA

SEQ ID NO: 11 - DNA, 666 bp, Escherichia coli
gacctggccagcctgagagcccagcagatcgagctggccagcagcgtgatcagagaggacaggctggacaaggacccccccgacctgatcgccggggccgatgtgggttttgagcagggcggcgaggtgaccagagccgccatggtgctgctgaagtaccccagcctggagctggtggagtacaaggtggccagaatcgccaccaccatgccctacatccccggcttcctgagcttcagagagtaccccgccctgctggccgcctgggagatgctgagccagaagcccgacctggtgttcgtggacggccacggcatcagccaccccagaagactgggcgtggccagccacttcggcctgctggtggacgtgcccaccatcggcgtggccaagaagagattgtgtggcaagttcgaacccctatccagcgagcccggcgccctggccccactgatggacaagggcgagcagctcgcctgggtgtggagaagcaaggccagatgcaaccccctgttcatcgccaccggccacagagtgagcgtggacagcgccttagcctgggtgcagagatgcatgaagggctacagactgcccgagcccaccagatgggccgacgccgtggccagcgagagacccgccttcgtgagatacaccgccaaccagccc

SEQ ID NO: 12 - protein, 222 aa, Escherichia coli
DLASLRAQQIELASSVIREDRLDKDPPDLIAGADVGFEQGGEVTRAAMVLLKYPSLELVEYKVARIATTMPYIPGFLSFREYPALLAAWEMLSQKPDLVFVDGHGISHPRRLGVASHFGLLVDVPTIGVAKKRLCGKFEPLSSEPGALAPLMDKGEQLAWVWRSKARCNPLFIATGHRVSVDSALAWVQRCMKGYRLPEPTRWADAVASERPAFVRYTANQP

SEQ ID NO: 13 - DNA, 843 bp, Escherichia coli
tacaccctgaactggcagcccccctacgactggagctggatgctgggcttcctggccgccagagccgtgagcggcgtggagaccgtggccgacagctactacgccagaagcctggccgtgggcgagtacagaggcgtggtgaccgccatccccgacatcgccagacacaccctgcacatcaacctgagcgccggcctggagcccgtggccgccgagtgcctggccaagatgagcagactgttcgacctgcagtgtaacccccagatagtgaacggggccctgggcaaactaggtgccgccagacccggtctgagactgcccggctgtgtggacgccttcgagcagggcgtgagagccatcctgggccagctggtgagcgtggccatggccgccaagctgaccagcagagtggcccagctgtacggcgagagactggacgacttccccgactacgtatgctttcctaccccccagagattggcggtggccgacttgcaggccctgaaggccctgggcatgcccctgaagcgtgcagaggccctgatccacctggccaatgccgcccttgaaggcacactgcctatgaccatccccggcgacgtggagcaggccatgaagaccctgcagaccttccccggcatcggcagatggaccgccaactacttcgccctgagaggctggcaggccaaggacgtgttcctgcccgacgactacctgatcaagcagagattccccggcatgacccccgcccagatcagaagatacgccgagagatggaagccctggagaagctacgccctgctgcacatctggtacaccgagggctggcagcccgacgaggcc

SEQ ID NO: 14 - protein, 281 aa, Escherichia coli
YTLNWQPPYDWSWMLGFLAARAVSGVETVADSYYARSLAVGEYRGVVTAIPDIARHTLHINLSAGLEPVAAECLAKMSRLFDLQCNPQIVNGALGKLGAARPGLRLPGCVDAFEQGVRAILGQLVSVAMAAKLTSRVAQLYGERLDDFPDYVCFPTPQRLAVADLQALKALGMPLKRAEALIHLANAALEGTLPMTIPGDVEQAMKTLQTFPGIGRWTANYFALRGWQAKDVFLPDDYLIKQRFPGMTPAQIRRYAERWKPWRSYALLHIWYTEGWQPDEA

SEQ ID NO: 15 - DNA, 471 bp, Methanobacterium barkeri
aagaagcagggcttcccccccgtgatcgacgagaacaccgagatcctgatcctgggcagcctgcccggcgacgtgagcatcagaaagcaccagtactacggccaccccggcaacgacttctggagactgctgggcagcatcatcggcgaggacctgcagagcatcaactaccagaacagactggaggccctgaagagaaacaagatcggcctgtgggacgtgttcaaggccggcaagagagagggcaacgaggacaccaagatcaaggacgaggagatcaaccagttcagcatcctgaaggacatggcccccaacctgaagctggtgctgttcaacggcaagaagagcggcgagtacgagcccatcctgagagccatgggctacgagaccaagatcctgctgagcagcagcggcgccaacagaagaagcctgaagagcagaaagagcggctgggccgaggccttcaagaga

SEQ ID NO: 16 - protein, 157 aa, Methanobacterium barkeri
KKQGFPPVIDENTEILILGSLPGDVSIRKHQYYGHPGNDFWRLLGSIIGEDLQSINYQNRLEALKRNKIGLWDVFKAGKREGNEDTKIKDEEINQFSILKDMAPNLKLVLFNGKKSGEYEPILRAMGYETKILLSSSGANRRSLKSRKSGWAEAFKR

SEQ ID NO: 17 - DNA, 885 bp, Unknown
  Yeast
aagctgaagagagagtacgacgagctgatcaaggccgacgccgtgaaggaaatcgccaaagaactgggcagcagacccctggaggtggccctgccggagaaatatatcgccagacacgaggagaagttcaacatggcctgcgagcacatcctggagaaggaccccagcctgttccccatcctgaagaacaacgagttcaccctgtacctgaaggagacccaggtgcccaacaccctggaggactacttcatcaggctggcaagcacgatattaagccagcagatcagcggccaggccgccgagagcatcaaggccagagtggtgagcctgtacggcggcgccttccccgactacaagatcctgttcgaggacttcaaggaccccgccaagtgcgccgaaatcgctaaatgtggtctgagcaagagaaagatgatctacctggagagcctggccgtgtacttcaccgagaaatataaggacatcgagaagctgttcggccagaaggacaacgacgaggaggtgatcgagagcctggtgaccaacgtgaagggcatcggcccctggagcgccaagatgttcctgatcagcggcctgaagagaatggacgtgttcgcccccgaggacctgggcatcgccagaggcttcagcaagtacctgagcgacaagcccgagctggagaaggagctgatgagagagagaaaggtggtgaagaagagcaagatcaagcacaagaagtacaactggaagatctacgacgacgacatcatggagaagtgcagcgagaccttcagcccctacagaagcgtgttcatgttcatcctgtggagactggccagcaccaacacggacgccatgatgaaggccgaggagaacttcgtgaagagc

SEQ ID NO: 18 - protein, 295 aa, Unknown
  Yeast
KLKREYDELIKADAVKEIAKELGSRPLEVALPEKYIARHEEKFNMACEHILEKDPSLFPILKNNEFTLYLKETQVPNTLEDYFIRLASTILSQQISGQAAESIKARVVSLYGGAFPDYKILFEDFKDPAKCAEIAKCGLSKRKMIYLESLAVYFTEKYKDIEKLFGQKDNDEEVIESLVTNVKGIGPWSAKMFLISGLKRMDVFAPEDLGIARGFSKYLSDKPELEKELMRERKVVKKSKIKHKKYNWKIYDDDIMEKCSETFSPYRSVFMFILWRLASTNTDAMMKAEENFVKS

SEQ ID NO: 19 - DNA, 23 bp, Artificial Sequence
  The sequence is synthesized.
cttccacatgagcgtggtcaggg

SEQ ID NO: 20 - DNA, 23 bp, Artificial Sequence
  The sequence is synthesized.
ggaccgcagccagcccggccagg

SEQ ID NO: 21 - DNA, 23 bp, Artificial Sequence
  The sequence is synthesized.
cacgttcaccttgccccacaggg

SEQ ID NO: 22 - DNA, 23 bp, Artificial Sequence
  The sequence is synthesized.
ggccccagtggctgctctggggg

SEQ ID NO: 23 - DNA, 23 bp, Artificial Sequence
  The sequence is synthesized.
aagttcgctaatcccggaactgg

SEQ ID NO: 24 - DNA, 23 bp, Artificial Sequence
  The sequence is synthesized.
taataattgatgtcatagattgg

SEQ ID NO: 25 - DNA, 23 bp, Artificial Sequence
  The sequence is synthesized.
gctcccatcacatcaaccggtgg

SEQ ID NO: 26 - DNA, 23 bp, Artificial Sequence
  The sequence is synthesized.
gctgcagaagggattccatgagg

SEQ ID NO: 27 - DNA, 23 bp, Artificial Sequence
  The sequence is synthesized.
gtgagtagagcggaggcaggagg

SEQ ID NO: 28 - DNA, 23 bp, Artificial Sequence
  The sequence is synthesized.
cgggcatcagaattccctggagg

SEQ ID NO: 29 - DNA, 23 bp, Artificial Sequence
  The sequence is synthesized.
caaagcaggatgacaggcagggg

SEQ ID NO: 30 - DNA, 23 bp, Artificial Sequence
  The sequence is synthesized.
ttcaatgtagacatctatgtagg

SEQ ID NO: 31 - DNA, 23 bp, Artificial Sequence
  The sequence is synthesized.
gcaggttaatgttacagccctgg

SEQ ID NO: 32 - DNA, 43 bp, Artificial Sequence
  The sequence is synthesized.
ggagtgagtacggtgtgccggagagcttcgtgctaaactggta

SEQ ID NO: 33 - DNA, 42 bp, Artificial Sequence
  The sequence is synthesized.
gagttggatgctggatggcagaggtaggtgccgctgtcattg

SEQ ID NO: 34 - DNA, 43 bp, Artificial Sequence
  The sequence is synthesized.
ggagtgagtacggtgtgccaacacatcggagagcttcgtgcta

SEQ ID NO: 35 - DNA, 42 bp, Artificial Sequence
  The sequence is synthesized.
gagttggatgctggatggtgaccacgctcatgtggaagtcac

SEQ ID NO: 36 - DNA, 38 bp, Artificial Sequence
  The sequence is synthesized.
ggagtgagtacggtgtgcagcaacctcaaacagacacc

SEQ ID NO: 37 - DNA, 40 bp, Artificial Sequence
  The sequence is synthesized.
gagttggatgctggatggtgcccagtttctattggtctcc

SEQ ID NO: 38 - DNA, 39 bp, Artificial Sequence
  The sequence is synthesized.
ggagtgagtacggtgtgcatgggagcagctggtcagagg

SEQ ID NO: 39 - DNA, 40 bp, Artificial Sequence
  The sequence is synthesized.
gagttggatgctggatggggttctggaaccacaccttcac

SEQ ID NO: 40 - DNA, 39 bp, Artificial Sequence
  The sequence is synthesized.
ggagtgagtacggtgtgcctttgggcggggtccagttcc

SEQ ID NO: 41 - DNA, 41 bp, Artificial Sequence
  The sequence is synthesized.
gagttggatgctggatggctctcttggagtgtctcctcatc

SEQ ID NO: 42 - DNA, 43 bp, Artificial Sequence
  The sequence is synthesized.
ggagtgagtacggtgtgcaaaacagtttgcattcatggagggc

SEQ ID NO: 43 - DNA, 43 bp, Artificial Sequence
  The sequence is synthesized.
gagttggatgctggatggtgaacaccagtgagtagagcggagg

SEQ ID NO: 44 - DNA, 43 bp, Artificial Sequence
  The sequence is synthesized.
ggagtgagtacggtgtgcgtggttccagaaccggaggacaaag

SEQ ID NO: 45 - DNA, 43 bp, Artificial Sequence
  The sequence is synthesized.
gagttggatgctggatgggtttgtggttgcccaccctagtcat

SEQ ID NO: 46 - DNA, 37 bp, Artificial Sequence
  The sequence is synthesized.
ggagtgagtacggtgtgcgtagcgcgcccactgcaag

SEQ ID NO: 47 - DNA, 43 bp, Artificial Sequence
  The sequence is synthesized.
gagttggatgctggatggttccaatcagtacgcagagagtcgc

SEQ ID NO: 48 - DNA, 41 bp, Artificial Sequence
  The sequence is synthesized.
ggagtgagtacggtgtgctttatttatgcacagggtggaac

SEQ ID NO: 49 - DNA, 37 bp, Artificial Sequence
  The sequence is synthesized.
gagttggatgctggatggaccagcatgttgcccacaa

SEQ ID NO: 50 - DNA, 38 bp, Artificial Sequence
  The sequence is synthesized.
ggagtgagtacggtgtgcatctcagcgctttcgtccac

SEQ ID NO: 51 - DNA, 38 bp, Artificial Sequence
  The sequence is synthesized.
gagttggatgctggatggctcatttccccactccctcc

SEQ ID NO: 52 - DNA, 43 bp, Artificial Sequence
  The sequence is synthesized.
ggagtgagtacggtgtgcccctcccttcaagatggctgacaaa

SEQ ID NO: 53 - DNA, 43 bp, Artificial Sequence
  The sequence is synthesized.
gagttggatgctggatggccactgtagtcacacagcaccagag

SEQ ID NO: 54 - DNA, 40 bp, Artificial Sequence
  The sequence is synthesized.
ggagtgagtacggtgtgccagcaaaccttcccttcactac

SEQ ID NO: 55 - DNA, 40 bp, Artificial Sequence
  The sequence is synthesized.
gagttggatgctggatggtcttgttccaccctgtgcataa

SEQ ID NO: 56 - DNA, 42 bp, Artificial Sequence
  The sequence is synthesized.
ggagtgagtacggtgtgcctgctcacttcattcctgcctcat

SEQ ID NO: 57 - DNA, 41 bp, Artificial Sequence
  The sequence is synthesized.
gagttggatgctggatggccatcatcgccctgacgtcaacc

SEQ ID NO: 58 - DNA, 18 bp, Artificial Sequence
  The sequence is synthesized.
ggagtgagtacggtgtgc

SEQ ID NO: 59 - DNA, 18 bp, Artificial Sequence
  The sequence is synthesized.
gagttggatgctggatgg